(19) United States
(12) Patent Application Publication
(10) Pub. No.: US 2020/0063205 A1
(43) Pub. Date: Feb. 27, 2020

(54) A METHOD FOR DIAGNOSING A MIGRAINE

(71) Applicant: Aalborg Universitet, Aalborg Øst (DK)

(72) Inventors: Parisa Gazerani, Aalborg Øst (DK); Meg Duroux, Gistrup (DK)

(21) Appl. No.: 16/488,198

(22) PCT Filed: Feb. 26, 2018
(86) PCT No.: PCT/EP2018/054671

§ 371 Date: Aug. 22, 2019

(30) Foreign Application Priority Data
Feb. 24, 2017 (DK) PA201770141

(51) Int. Cl. C12Q 1/6883

(52) U.S. Cl. CPC C12Q 1/6883; C12Q 2600/178; C12Q 2600/158

(57) ABSTRACT

The invention relates to a method of diagnosing a migraine and/or migraine attack, the method in a subject comprising determining a change in the expression level of a biological marker in the biological sample. Preferably, the method is the in vitro method.

### TECHNICAL FIELD

The invention relates to new methods for diagnosing migraine. Particularly the invention relates to the methods for diagnosing migraine by biomarker(s) profiling.

### BACKGROUND ART

Migraine is a common neurobiological, primary headache disorder with recurrent attacks, characterized by different phases, symptoms and frequency that vary among patients. In general, those with migraine have about one attack per month, often initiated at puberty and most burdensome in the productive years until regression or termination. It affects two to three times more women than men. Additionally, migraine is linked with anxiety, depression and increase risk of stroke, cardiovascular diseases, and epilepsy among others. The first premonitory phase occurs hours to days before the attack in half of the patients with changes in mood, tiredness or sensitivity to sensory input such as noise. The next phase starts minutes to an hour before the headache attack, and includes reversible neurological visual symptoms (aura) only manifested in 30% of affected individuals (Kojić, 2013). A subset of those patients with aura has sensory/speech disturbances or motor weakness, which are less common (Russel et al., 2002). The headache phase is the most disabling, lasting up to two days if untreated, with moderate to severe pulsating pain locating in any region of the head, face or neck, which often presents as one-sided. The pain aggravates by hard physical activity (Varkey, 2011) and is associated with nausea/vomiting, light, smell, and noise sensitivity (Evans, 2014). Following headache, the resolution phase can take one further day where patients recover and might feel tired or depressed. There are several factors that patients relate to cause migraine attack of which stress and female hormones are the most widespread, but also sleep disturbances, physical exhaustion and particular food or odor are known as triggers. The International Headache Society (IHS) has hierarchically classified headaches as primary or secondary headaches. Primary headaches are not symptoms of a disease or disorder and are considered disorders on their own. Migraine is classified as primary headache, divided into two major sub-types of migraine without aura (MO) and migraine with aura (MA). There are further classification codes to describe additional important features, e.g. childhood migraine or the sensory/motor weakness in migraineurs with aura. More details are available on the IHC version 3 beta available at the IHS web-page: http://ihs-classification.org.

A major obstacle is the lack of diagnosis or misdiagnosis of the condition. Besides low levels of awareness and knowledge among health-care providers, the present classification system by IHS is insufficient since it is solely based upon patient history of symptoms and professional interview, which are highly subjective. Some of the symptoms might resemble other primary or secondary headache disorders, e.g. infrequent episodic tension-type headache resembles migraine without aura and familial hemiplegic migraine is often mistaken for epilepsy (http://ihs-classification.org).

Migraine is a cluster of different phenotypical subtypes rather than a single disorder with clinical homogenous features and natural history varying from one patient to another including triggering factors.

Furthermore, patients might be diagnosed with more than one subtype of migraine according to several other classification levels. There is a need for reliable diagnostic markers of migraine, as well as migraine attacks as well as patent stratification to follow response to medication.

### Objectives of the Present Invention

The present inventors have now realized the need for improvement of existing diagnostic tools, and in particular, for performing diagnosis based on the biological markers, and in particular, for in vitro diagnostic methods.

Accordingly, the inventors also realized a need for new biomarkers less vulnerable to degradation than the existing biomarkers.

It has been also realized that there is a need to provide a fraction of the biological sample wherein the biomarker is present in the increased quantities.

### SUMMARY OF THE INVENTION

In one aspect, the invention provides a method of diagnosing migraine in a subject comprising: providing an isolated biological sample obtained from the subject, determining in said isolated biological sample the level of at least one biomarker expression, providing a control level for said at least one biomarker expression determined as a base line level of said biological marker expression in healthy subjects, comparing said level with said control level for at least one biomarker expression, indicating that the subject is likely to have migraine when said level of at least one biomarker expression in said isolated biological sample is higher than said control level for said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, and IGHV4-34 and indicating that the subject is unlikely to have migraine when said level of at least one biomarker expression in said isolated biological sample is lower than or equal to said control level for said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, and IGHV4-34.

In the further embodiment, the invention provides the method of diagnosing migraine in a subject, wherein the biomarker is selected from the group APOC4, DSC1, DSP.

In the further embodiment, the invention provides the method of diagnosing migraine, wherein the healthy subjects are matched with the subjects by age and/or sex.

In the further aspect, the invention provides a method of diagnosing migraine attack in a subject having migraine comprising: providing an isolated biological sample obtained from the subject, determining in said isolated biological sample the level of at least one biomarker expression, providing a reference level for said at least one biomarker expression, determined as a base level of said biomarker expression in the subject in a pain-free period, comparing said level with said reference level of at least one biomarker expression, indicating that the subject is likely to have a migraine attack when the level of said at least one biomarker expression in said isolated biological sample is higher than said reference level for said at least one biomarker selected from the group consisting of hsa-miR-140-3p-4395345, hsa-miR-184-4373113, hsa-miR-195-4373105, hsa-miR-324-3p-4395272, hsa-let-7b-4395446, rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), hsa-miR-10b #-002315 (FAM,NFQ), and/or expression in said isolated biological sample is lower than said reference level for said at least one biomarker selected from the group consisting of hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV, desmocollin-1, and desmoplakin, and the subject is unlikely to have a migraine attack when the level of at least one biomarker expression in said isolated biological sample is lower than or equal to said reference level for said at least one biomarker selected from the group consisting of hsa-miR-140-3p-4395345, hsa-miR-184-4373113, hsa-miR-195-4373105, hsa-miR-324-3p-4395272, hsa-let-7b-4395446, rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), and hsa-miR-10b #-002315 (FAM,NFQ), and/or the level of at least one expression in said isolated biological sample is higher than or equal to said reference level for said at least one biomarker selected from the group consisting of hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV, desmocollin-1, and desmoplakin.

In the further embodiment of the invention it is provided the method of diagnosing migraine attack in a subject having migraine the method, wherein the biomarker is selected from the group APOC4, DSC1, DSP.

In the further embodiment, the invention provides the method of diagnosing migraine attack in a migraine subject, wherein said migraine attack is migraine attack with an aura.

In the further embodiment, the invention provides the method of diagnosing a migraine in a subject comprising: providing an isolated biological sample obtained from the subject comprising predominantly human exosomes fraction, determining in said isolated biological sample the level of at least one biomarker expression, providing a control level of said at least one biomarker expression, said control level is a base line level of said biological marker expression in healthy subjects, comparing said level of the at least one biomarker expression with said control level of said at least one biomarker expression, indicating that the subject is likely to have a migraine with an aura when said at least one biomarker expression level in said isolated biological sample is higher than said control level for said at least one biomarker selected from miR-122 and miR-885-5p, and/or said at least one biomarker expression level in said isolated biological sample is lower than said control level for said at least one biomarker selected from miR-135b, miR-129-3p and miR-146a and indicating that the subject is unlikely to have a migraine with an aura when said level of at least one biomarker expression is lower than or equal to said control level for said at least one biomarker selected from miR-122 and miR-885-5p, and/or said at least one biomarker expression level in said isolated biological sample is higher than or equal to said control level for said at least one biomarker selected from miR-135b and miR-146a.

In the further aspect, the invention provides the method of determining a predisposition to develop migraine in a subject comprising: providing an isolated biological sample obtained from the subject, determining in said isolated biological sample the level of at least one biomarker expression, providing a control level for said at least one biomarker expression determined as a base line level of said biological marker expression in healthy subjects, comparing said level with said control level for at least one biomarker expression, indicating that the subject is likely to develop migraine if said expression level in said isolated biological sample is higher than said control level for said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34; miR-122 and miR-885-5p and/or said at least one biomarker expression level in said isolated biological sample is lower than said control level for said at least one biomarker selected from miR-135b and miR-146a and indicating that the subject is unlikely to develop migraine if said expression level in said isolated biological sample is equal to or lower than said control level for said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34; miR-122 and miR-885-5p and/or said at least one biomarker expression level in said isolated biological sample is equal to or higher than said control level for said at least one biomarker selected from miR-135b and miR-146a.

In the further aspect, the invention provides the method of determining a predisposition to develop migraine in a subject, wherein the biomarker is selected from the miR-122, miR-146a, miR-122, miR-885-5p, APOC4, DSC1, DSP.

In the further embodiment, the invention provides the method, wherein a difference between said level of said at least one biomarker expression and said reference level or said control level of said at least one biomarker expression is significant as determined by p-value less than 0.05.

In the further embodiment, the invention provides the method according to any previous embodiments, wherein the difference between said level of said at least one biomarker expression and said reference level or said control level of said at least one biomarker expression is considered significant when the change in the expression level is at least two-fold.

In the further embodiment, the invention provides the method according to any of the previous embodiments, wherein said isolated biological sample is selected from blood plasma, serum, urine, saliva.

In the further embodiment of the invention, said biological sample comprises extracellular vesicles of 50-20000 nm, preferably serum exosomes with a diameter between 30 nm and 120 nm, preferably serum exosomes with a diameter 100 nm.

In the further embodiment of the invention, the subject is a human.

In the further embodiment of the invention, said method is an in vitro method.

In the further aspect, the invention provides a molecular biomarker for or use in diagnosing a migraine in a patient, wherein said biological compound is selected from APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34.

In the further aspect, the invention provides a molecular biomarker for use in diagnosing a migraine attack in a migraine patient, wherein said biological compound is selected from the group consisting of hsa-miR-140-3p-4395345 (FAM,NFQ), hsa-miR-184-4373113 (FAM,NFQ), hsa-miR-195-4373105 (FAM,NFQ), hsa-miR-324-3p-4395272 (FAM,NFQ), hsa-let-7b-4395446 (FAM,NFQ), rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), hsa-miR-10b #-002315 (FAM,NFQ), hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV, desmocollin-1, and desmoplakin.

### BRIEF DESCRIPTION OF THE DRAWINGS

FIG. 1. Table 1. A—Up-regulated miRNAs migraine attack with aura cohort 1. B—Down regulated miRNAs migraine attack with aura cohort 1.

FIG. 2. A graphical representation of the study design.

FIG. 3. Significantly deregulated exosomal proteins.

A. Volcano plot of significance (y-axes) versus fold-change (x-axes) of proteomic data using Perseus v. 1.5.2.6. The differences in exosomal protein expression during headache attack was compared to pain-free period of migraine patients, cohort 1 (n=8). The cut off curve indicates proteins-of-interest. Points above the curve have p-values <0.05. Gray points: fold-change less than 2 (log 2=1). Negative x-values indicates fold downregulation.

B. Identified candidate proteins by a Volcano plot based strategy, with false detection rate (FDR)=0.05 and s0=0.01. Seq. Cov.=sequence coverage—with large sequence coverage, the more certain is the identification of the protein.

FIG. 4. A list of unique proteins found in exosomes from Migraine patients (cohort 1) compared to healthy serum.

FIG. 5. A. Diagram with number of unique and common proteins in serum exosomes of migraine patients compared to serum of healthy controls. B. Top 10 most abundance proteins ranked after iBAQ values in serum exosomes of migraine patients, cohort 1 (n=8) and serum of healthy control (n=1). iBAQ=Intensity-based absolute quantification. UniProt Acc.=unique identifier used to cite UniProt entries.

FIG. 6. Particle concentration (y-axis) and particle size distribution (x-axis) of isolated exosomes in serum of migraine patients (n=6) and healthy controls (n=6) by NTA. Dotted curves=duplicate samples, solid curves (average). The vesicles suspension was diluted 1:50 before analysis. Mode, mean particle size (nm) and particle concentration in particles/mL (P/mL) are specified.

FIG. 7. Fold changes of candidate miRNAs of migraine patients, cohort 2, pain-free period compared to healthy control. With adjusted p-values<0.006.Mr=fold change, SD=standard deviation of fold change.

### DETAILED DESCRIPTION

At the core of the present inventions stands the inventor's realization that certain types of miRNAs and proteins can be used to diagnose migraine in patients as well as to diagnose migraine attacks in migraine patients or to indicate that the subject is likely to develop a migraine or migraine attack. The new diagnostic methods also allow distinguishing migraine from other types of headaches.

Accordingly, the invention provides new biomarkers less vulnerable to degradation than the existing biomarkers. The new biomarkers at the same time are more sensitive with respect to levels required for analysis.

The invention also provides a fraction of the biological sample wherein the biomarker is present in the increased quantities.

Various aspects and embodiments of the invention are described in further detail herein.

### The Complex Pathophysiology of Migraine

Current theory attributes to a dysfunction in the brain as the mechanism of emotional and sensory symptoms presented in the premonitory phase of migraine, while the aura phase is explained as a slow cortical spreading depression (CSD), starting in the visual or somatosentory cortex leading to visual or sensory disturbances. In the headache phase, it is generally accepted that pain is generated by an activation of the trigeminal nerve terminals, which transmit nociceptive inputs from large cranial and meningeal vessels, the few pain sensitive structures in the brain. Afferent fibers' excitation releases several inflammatory neuropeptides such as substance P (SP) and calcitonin gene-related peptide (CGRP) in the peripheral nervous system including trigeminal ganglion, leading to dilation of meningeal blood vessels; plasma leakage and mast cell activation, which collectively lead to release of diverse range of pro-inflammatory mediators (cytokines, serotonin, prostaglandin (PGE2) and bradykinin) maintaining the initial inflammatory response (Tajti, 2009). This phenomenon, called neurogenic inflammation is considered to prolong/sustain the pain and enhance peripheral sensitization, so innocuous mechanical changes as the pulse activates the nociceptive nerves, leading to the throbbing pain sensation which worsen during hard physical activity (Varkey, 2011).

Furthermore, the enhanced peripheral signals disturb the chemical balance and increase glutamate level in second order neurons in the brain stem (the trigeminal nucleus caudalis (TNC), and in the thalamus, leading to neuronal hyperexcitability and thereby central sensitization. This hyperexcitability might lead to functional plasticity, where non-noxious sensory stimuli of the skin/scalp leads to facial/scalp allodynia (perception of pain) (Pietrobon, 2013). Activation of projections from thalamic neurons to other brain areas likely contributes to the other neurological symptoms accompanying the headache and resolution phase (Pietrobon, 2013).

However, the periodic initiation of brain dysfunction in the premonitory phase, activation of CSD and trigeminal nerve terminals are still not very well investigated.

### Satellite Glial Cells Involvement in Pain

Increasing evidence implicates that dysfunction of glia cells is a key mechanism in development and maintenance of chronic pain. Especially, microglia and astrocyte of the central nervous system (CNS) and satellite glial cells (SGCs) of the peripheral nervous system (PNS) are associated with painful syndromes (Ji, 2013). In the peripheral nervous system, satellite glial cells wrap around the neurons in the sensory ganglia and hence make a very unique unit of SGC-neuron that is now known as an important structure in pain signaling pathways due to the close neuro-glial interactions. SGCs are supportive non-neuronal cells, which wrap around each neuron within dorsal root and trigeminal ganglia, allowing close bidirectional interaction between neurons and glia (neuron-glia) and also glial cells (glia-glia), facilitating the maintenance of neuronal homeostasis (Hanani, 2005). They are proposed to play a key role in pain, both in initiation and chronification under neuropathic and inflammatory conditions, based on their ability to influence the neuronal excitability (Jasmin, 2010). This phenomenon is partly mediated by extensive proliferation (Hanini, 2005), interconnections and cross talk of SGCs after nerve injury or inflammation, which is collectively thought to influence the excitability of adjacent neurons and subsequently modulation of nociception pathways (Ohara, 2009). Expressional changes of nociceptive related proteins in the SGCs and release of pro-inflammatory cytokines have been suggested to affect the firing rate of the neurons and further modulation of nociception (Ji, 2013). It has been shown that injury or inflammation activates SGCs with increased glial marker expression of glial fibrillary acidic protein (GFAP) (Costa, 2015). The sensory perception is enhanced due to extensive proliferation, glial interconnection and crosstalk of neighboring SGCs which together influence the excitability of nearby neurons (Ohara, 2009). Furthermore, upregulation of adenosine triphosphate, chemokine and Toll-like receptors in SGCs induces release of pro-inflammatory cytokines such as, TNF-alfa, PGE2 and NO, in addition to growth factors and proteases (Ji, 2013). SGCs also affect neuronal excitability by 1) releasing of IL-beta from SGCs, which suppresses voltage dependent K+ channels, and 2) reducing of expression of inward rectifying potassium channel (Kir4.1) on SGCs, which enhances neuronal K+ level and thereby neuronal firing rate (Ohara, 2009; Costa, 2015). The extensive changes in proteins of neurotransmitters, signaling molecules, ion-channels and structural proteins in the pain pathway are post transcriptionally regulated by a class of small non-coding RNAs, known as microRNAs (miRNAs) (Etheridge, 2011).

### Extracellular Vesicles

Extracellular vesicles are lipid-enclosed round shaped membranes within the nanosize range, which are shed by a variety of cells. They are a heterogeneous population of vesicles with different origin, membrane composition and contents (Yánez-Mó, 2015). The smallest vesicles, known as exosomes, these are formed in a subset of late endosomes, called multivesicular bodies (MVBs) by inward budding of the limiting membrane (Gupta, 2014). They range in size, generally from 30-100 nm as seen by electron microscope, and up to 150 nm (by Nanoparticle Trackting Analysis, NTA) (Colombo, 2014) and contain a cargo of nucleic acid, including selected pre-synthesized miRNAs, different from the intracellular miRNAs. Besides nucleic acid, exosomes contains different proteins. Some proteins are common to most exosomes, e.g. cytoskeletal proteins (actin, tubulin) and membrane bound proteins linked with their endosomal origin, e.g. adhesion molecules (tetraspanins, integrins), and transport and fusion proteins (Rab, annexins). Other cellular proteins might be specific for different cell types. The exosomes are stored within the MVB until degradation in lysosomes or released into the extracellular space in pulses (Cocucci, 2015). In contrast, a random load of cell fragments from the cytoplasm is bud of from the plasma membrane in 50-2,000 nm sized microvesicles without delay (Akers, 2013). The two types of extracellular vesicles have several similarities in size and composition, both types are relevant in the context of the invention, while exosomes are preferred.

There is evidence that exosomes are taken up by target cells, where their cargo of proteins, miRNAs and mRNA affect the cell functionality, however studies has been performed in immune, cancer and stem cells (Gupta, 2014).

None of the studies dealt with the miRNA were concerned with their involvement in the mechanism underlying migraine pain.

### MicroRNA Modulates Protein Expression

Mature miRNAs are single-stranded molecules of 19-24 nucleotides, surprisingly well conserved through evolution and expressed among a variety of species in a number correlated to the complexity of developmental program. Thus, the number of human miRNAs entries in miRBase is 1,881 precursors and 2,588 mature, whereas Norwegian rat, Rattus norvegicus has 495 precursors and 765 mature (http://www.mirbase.org, 16 May 2016). MicroRNAs are synthesized in a way similar to that of proteins. Firstly, a sequence of 100 nucleotides or more is generated, termed a primary miRNA transcript (pri-miRNA) in the nucleus, catalyzed by RNA polymerase II or III (Etheridge, 2011). Post-transcriptionally, the pri-mi RNA is modified by adding a 3′ poly-A tail and 5′ cap and processed/folded by the enzyme Drosha to a 70 nucleotid long hairpin, named precursor-RNA (pre-miRNA). After transportation by exportin 5 to the cytoplasm, another enzyme, Dicer cleaves the double-stranded RNA to the mature microRNA of 19-24 nucleotides (Etheridge, 2011). A RNA-induced silencing complex (RISC) incorporates one stand of mature miRNA, which acts as a template, that partially complements within the 3′ untranslated region of a messenger RNA (mRNA) or in the reading frame, thereby represses or initiates cleavage of the mRNA and decreases the protein production (Kynast, 2013).

### Gaps in Current Migraine Classification

Currently, there is lack of diagnosis or misdiagnosis. Beside low levels of awareness and knowledge among health-care providers, the present classification system by IHS is insufficient since it is solely based upon patient history of symptoms and professional interview, which are highly subjective. Some of the symptoms might resemble other primary or secondary headache disorders, e.g. infrequent episodic tension-type headache resembles migraine without aura and familial hemiplegic migraine is often mistaken for epilepsy (http://ihs-classification.org). Even with correct diagnosis, a patient within the same classification might respond differently to medication, and the same person can have different symptoms, frequency and severity from attack to attack over time. Furthermore, patients might be diagnosed with more than one subtype of migraine and several other classification levels, which make treatment strategies even more complicated. Besides, there is no clinical maker for treatment follow-up (Loder, 2002) to show patients who are an advantage over a time course of treatment.

### Mechanism-Based Biomarkers in Migraine

The most commonly used classification is an IHS classification for headaches, wherein migraine headaches are included as a subsection. It can be found on the www.ichd-3.org web site and the most recent version is the 3^{rd} edition (Beta version) which can be found on http://www.ihs-classification.org/_downloads/mixed/International-Headache-Classification-III-ICHD-III-2013-Beta.pdf.

However, this classification is not based on objective criteria such as biomarkers and mainly is based on the criteria that should be checked by a neurologist or specialist in an interview with patients, which can be highly subjective. For instance, pain is classified as chronic versus acute or inflammatory versus neuropathic.

Given the limitations in the present clinical classification system of pain and in particular for headaches, the inventors focused on discovery of reliable objective measurements, associated with the pathophysiology of migraine (biomarkers), that are able to diagnose migraine. Several studies have used knowledge-based approaches to analyze biofluids, particularly blood for biomarker candidates based on known molecules involved in the pathophysiology of migraine.

However, protein-based biomarkers measured with ELISA are vulnerable to degradation, often with short half-lives given inconsistency and low evidence (specificity and sensitivity). The present invention overcomes this by providing biomarkers with the improved resistance to degradation and increased sensitivity.

Transcriptomic (miRNA)

The study of miRNAs received a marked increase in attention in the field of biomarker discovery after their regulatory role in protein synthesis and possible implication in disease was first identified in the early 2000s. Deregulated miRNAs in cancer, infection and cardiovascular diseases have intensely been studied for potential biomarker and therapeutic targets, whereas identification of miRNAs in pain related processes has not been well investigated.

### Proteomic Analysis

In general, large-scale proteomic analysis called “shotgun proteomics” is performed by tandem mass spectrometry (MS/MS) using a peptide-based approach with label-free intensity-based quantification. However, body fluids are highly complex and detection of specific low abundance candidate proteins and miRNA biomarkers might be hampered by the more abundant molecules, e.g. the 5 most abundance proteins in plasma represent 95% of the total proteins (Frantzi, 2014). The problem is overcome in accordance with the invention by providing the biomarkers according to the invention.

### Exosomes in Biomarker Discovery

In ExoCarta, a comprehensive database of identified exosomal molecules, there are currently 9,769 proteins and 2,838 miRNAs from 286 studies (http://exocarta.org, 14 May 2016). However, when it comes to disorders of pain, only two studies have addressed miRNAs in extracellular vesicles, of which only one has identified four altered miRNAs that were significantly altered (miR-181a, let-7b, miR-27b, miR-22) in serum-exosomes of 15 female migraine patients without aura (MO) compared with healthy controls (Tafuri, 2015). So far, no proteomic study has been conducted in this field.

A method of diagnosing a migraine in a subject is provided. Initially, an isolated biological sample obtained previously from the subject is provided. The biological sample can be any sample wherein the biomarkers according to the invention are present and isolated either directly from the sample, such as serum or from the subtraction of the sample; for instance exosomes, with the techniques described in the art. The biological sample can be but not limited to blood plasma, serum, urine, saliva. The method according to the present invention is performed in vitro.

It is further disclosed that in the method of diagnosing migraine in the isolated biological sample the level of at least one biomarker expression is determined. The level is the level of the expression of the at least one biomarker in the biological sample of the subject. By the way of an example exosomes were isolated by ultracentrifugation and the expression level was analysed by quantitative real time PCR, as detailed in Example 1. However, it shall be understood that other known techniques suitable for the purpose can be used without departing from the scope of the invention.

Further, a control level for said at least one biomarker expression determined as a base line level of said biological marker expression in healthy subjects is provided in accordance to this aspect of the invention. For determining the base level of the biomarker expression the samples are preferably matched by gender and/or age to the subject tested. The base level can also be determined in the healthy subjects matched to the tested subject by race. The base level can be determined based on the samples from at least two healthy subjects in accordance with the invention, preferably at least three healthy subjects. Preferably, the base level is determined based on the samples from a plurality of healthy subjects, such as between three and twelve healthy subjects, most preferably between three and eight.

It is indicated in accordance with the invention that the subject is likely to have migraine or it is indicated that the subject is more likely to develop migraine when the level in the said isolated biological sample is higher than said control level for said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, and IGHV4-34.

It is also indicated in accordance with the invention that the subject is unlikely to have migraine when said level of at least one biomarker expression in said isolated biological sample is lower than or equal to said control level for said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, and IGHV4-34.

The reference to the unique identifier in the NCBI database for each protein is provided in Table 2, FIG. 4. Typically, the control level can be, preferably about zero, as these proteins are uniquely expressed in the migraine patients. The change in the expression between the control level and the level in the biological sample can indicate that the subject is more likely to have migraine if the increase in the expression in a subject is at least two-fold. Accordingly, if there is no change in the expression level of the proteins according to the invention or the change is less than two-fold it can be indicated that the subject is unlikely to have migraine.

In a further embodiment of the invention the healthy subjects are matched with the tested subjects by age/sex. This is to improve accuracy of the comparison.

In a further embodiment of the invention a method of diagnosing a migraine attack in a subject having migraine is provided. In accordance with the invention the distinction can be made between different types of headaches, and in particular the migraine attach headache can be differentiated from the other types.

Further in the method it is provided an isolated biological sample obtained from the subject diagnosed with migraine. The isolated biological sample can be obtained from the subject by the techniques known in the art, however they are not encompassed by the invention, since the invention, preferably, relates to the in vitro determination of the expression level.

In accordance with the invention the biological sample can be processed by, for example, ultracentrifugation or other exosome isolation methods that are state of the art followed by proteomic analysis such as mass spectrometry or ELISA for determining protein expression and PCR for determining the expression level TaqMan® Array screen for miRNAs.

Further the level of at least one biomarker expression is determined in the isolated biological sample. Hence, the expression level for the same biomarker is compared during migraine attack and a reference level determined in a pain-free period. As surprisingly discovered by the inventors, the biomarkers according to the invention can have different levels of expression in a pain free period and during the migraine attacks.

Further the level of at least one biomarker expression is compared with the reference level of the same biomarker expression. As disclosed above, the level of the biomarker expression is the expression level in the test subject.

It is indicated that the subject is likely to have the migraine attack when the expression level in said isolated biological sample is higher than said reference level for said at least one biomarker selected from the group consisting of hsa-miR-140-3p-4395345, hsa-miR-184-4373113, hsa-miR-195-4373105, hsa-miR-324-3p-4395272, hsa-let-7b-4395446, rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), and hsa-miR-10b #-002315 (FAM,NFQ), and/or expression in said isolated biological sample is lower than said reference level for said at least one biomarker selected from the group consisting of hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), and the proteins apolipoprotein C-IV, desmocollin-1, desmoplakin.

It is further indicated that the subject is unlikely to have a migraine attack when the level of at least one biomarker expression in said isolated biological sample is lower than or equal to said reference level for said at least one biomarker selected from the group consisting of hsa-miR-140-3p-4395345, hsa-miR-184-4373113, hsa-miR-195-4373105, hsa-miR-324-3p-4395272, hsa-let-7b-4395446, rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), and hsa-miR-10b #-002315 (FAM,NFQ), and/or the level of at least one expression in said isolated biological sample is higher than or equal to said reference level for said at least one biomarker selected from the group consisting of hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV, desmocollin-1, and desmoplakin

The miRNA sequences are freely available from http://microrna.sanger.ac.uk/ Database and are identified by the following accession numbers:

Hsa-miR-140-3p-4395345 (FAM,NFQ), Accession No: MIMAT0004597; hsa-miR-184-4373113 (FAM,NFQ) Accession No: MI0000481; hsa-miR-195-4373105, (FAM,NFQ) Accession No: MI0000489; hsa-miR-324-3p-4395272 (FAM,NFQ) Accession No: MIMAT0000762; hsa-let-7b-4395446, (FAM,NFQ) Accession No: MI0000063, rno-miR-7#-001338 (FAM,NFQ) Accession No: MI0000641, hsa-miR-223#-002098 (FAM,NFQ), Accession No: MI0000300, hsa-miR-942-002187 (FAM,NFQ), Accession No: MI0005767, hsa-miR-1260-002896 (FAM,NFQ), Accession No: MI0006394, hsa-miR-34a-4395168 (FAM,NFQ), Accession No: MI0000268; hsa-miR-185-4395382 (FAM,NFQ), Accession No: MI0000482; hsa-miR-193a-5p-4395392 (FAM,NFQ), Accession No: MIMAT0004614; hsa-miR-224-4395210 (FAM,NFQ), Accession No: MI0000301; hsa-miR-340-4395369 (FAM,NFQ), Accession No: MI0000802; hsa-miR-522-4395524 (FAM,NFQ), Accession No: MI0003177; hsa-miR-579-4395509 (FAM,NFQ), Accession No: MI0003586; hsa-miR-511-4373236 (FAM,NFQ), Accession No: MI0003127; dme-miR-7-000268 (FAM,NFQ), Accession No: MI0000127; hsa-miR-10b #-002315 (FAM,NFQ), Accession No: MI0000267; and/or expression in said isolated biological sample is lower than said reference level for said at least one biomarker selected from the group consisting of hsa-let-7c-4373167 (FAM,NFQ), Accession No: MI0000064; hsa-let-7e-4395517 (FAM,NFQ), Accession No: MI0000064; hsa-miR-28-3p-4395557 (FAM,NFQ), Accession No: MIMAT0004502; hsa-miR-199a-3p-4395415 (FAM,NFQ), Accession No: MIMAT0000232, hsa-miR-323-3p-4395338 (FAM,NFQ), Accession No: MIMAT0000755; hsa-miR-363-4378090 (FAM,NFQ), Accession No: MI0000764; hsa-miR-367-4373034 (FAM,NFQ), Accession No: MI0000775; hsa-miR-346-4373038 (FAM,NFQ), Accession No: MI0000826; hsa-miR-425-4380926 (FAM,NFQ) Accession No: MI0001448, hsa-miR-454-4395434 (FAM,NFQ) Accession No: MI0003820, hsa-miR-628-5p-4395544 (FAM,NFQ) Accession No: MIMAT0004809, hsa-miR-206-000510 (FAM,NFQ) Accession No: MI0000490, hsa-miR-572-001614 (FAM,NFQ) Accession No: MI0003579, hsa-miR-939-002182 (FAM,NFQ) Accession No: MI0005761, hsa-miR-19b-1#-002425 (FAM,NFQ) Accession No: MI0000074, and hsa-miR-628-3p-002434 (FAM,NFQ) Accession No: MIMAT0003297.

In order to indicate that the subject is more likely to have a migraine attack at least one of the following miRNAs as the following changes in the expression pattern. hsa-miR-140-3p-4395345 (FAM,NFQ) is upregulated at least 2 times, preferably at least 2.06 times. hsa-miR-184-4373113 (FAM,NFQ) is upregulated at least 2 times, preferably at least 10 times, more preferably at least 50 times, more preferably at least 100 times, more preferably at least 150 times, even more preferably at least 177 times. hsa-miR-195-4373105 (FAM,NFQ) is upregulated at least 2 times, preferably at least 2.15 times. hsa-miR-324-3p-4395272 (FAM,NFQ) is upregulated at least 2 times, more preferably at least 3 times, more preferably at least 3.78 times. hsa-let-7b-4395446 (FAM,NFQ) has been upregulated at least 2 times, more preferably at least 2.5 times. rno-miR-7#-001338 (FAM,NFQ) is upregulated at least 2 times, more preferably at least 2.48 times. hsa-miR-223#-002098 (FAM,NFQ) is upregulated at least 2 times, preferably at least 2.33 times. hsa-miR-942-002187 (FAM,NFQ) is upregulated at least 2 times, more preferably at least 4 times, more preferably at least 8 times, more preferably at least 8.6 times. hsa-miR-1260-002896 is upregulated at least 2 times, preferably at least 2.5 times, more preferably at least 2.86 times. hsa-let-7c-4373167 (FAM,NFQ) is downregulated at least two times, preferably at least 2.39 times. hsa-let-7e-4395517 (FAM,NFQ) is downregulated at least 2 times, preferably at least 3 times, more preferably at least 3.14 times. hsa-miR-28-3p-4395557 (FAM,NFQ) is downregulated at least two times, preferably at least 2.93 times. hsa-miR-199a-3p-4395415 (FAM,NFQ) is downregulated at least 2 times, preferably at least 2.11 times. hsa-miR-323-3p-4395338 (FAM,NFQ) is downregulated at least two times, preferably at least 2.98 times. hsa-miR-363-4378090 (FAM,NFQ) is downregulated at least two times, preferably at least 2.5 times more preferably at least 2.75 times. hsa-miR-367-4373034 (FAM,NFQ) is downregulated at least two times, preferably at least 4 times, more preferably at least 10 times, more preferably at least 15 times, more preferably at least 18 times. hsa-miR-346-4373038 (FAM,NFQ) is downregulated at least two times, preferably at least 2.6 times. hsa-miR-425-4380926 (FAM,NFQ) is downregulated at least two times, preferably at least 2.13 times. hsa-miR-454-4395434 (FAM,NFQ) is downregulated at least 2 times, preferably at least 10 times, more preferably at least 50 times, more preferably at least 100 times, more preferably at least 200 times, more preferably at least 300 times, more preferably at least 400 times, more preferably at least 500 times, more preferably at least 600 times, more preferably at least 700 times, more preferably at least 800 times, more preferably at least 900 times, more preferably 1000 times, more preferably at least 1100 times, more preferably at least 1200 times, more preferably at least 1300 times, more preferably at least 1400 times. hsa-miR-628-5p-4395544 (FAM,NFQ) is downregulated at least 2 times, preferably at least 2.5 times, more preferably at least 2.78 times. hsa-miR-206-000510 (FAM,NFQ) is downregulated at least 2 times, preferably at least 4 times, more preferably at least 6 times, more preferably at least 8 times, more preferably at least 10 times, more preferably at least 12 times, more preferably at least 14 times, more preferably at least 20 times, more preferably at least 25 times, more preferably at least 30 times, more preferably at least 35 times, more preferably at least 40 times, more preferably at least 41 times. hsa-miR-939-002182 (FAM,NFQ) is downregulated at least 2 times, more preferably at least 4 times, more preferably at least 6 times, more preferably 6.62 times. hsa-miR-628-3p-002434 (FAM,NFQ) is downregulated at least 2 times, preferably at least 3 times. When the changes in up/or downregulation of the biological markers above are not detectable or less than two fold, it can be indicated in accordance with the invention that the subject is unlikely to have the migraine attack.

In order to indicate that the subject is more likely to have the migraine if: apolipoprotein C-IV is downregulated compare to the reference level at least 2 times, preferably at least 4 times, more preferably at least 6 times, more preferably at least 7 times; desmoplakin is downregulated compare to the reference level at least 2 times, more preferably at least 3.5 times, desmocollin-1 is downregulated at least 2 times, more preferably at least 2.5 times.

If the changes in the expression level of apolipoprotein C-IV, desmoplakin and desmocollin-1 are undetectable or less than two-times, it can be indicated in accordance with the invention that the subject is unlikely to develop a migraine attack.

These miRNA and proteins have been discovered by the inventors to demonstrate a difference in expression pattern during the pain-free period and during migraine attack.

Preferably, the biological sample is serum, preferably serum exosomes. The biological sample is obtained by ultracentrifugation to enrich the exosome fraction; the supernatant discharged and the remaining pellet resuspended in a suitable diluent such as PBS.

### Serum Biomarkers of Migraine and Migraine Pain Headache

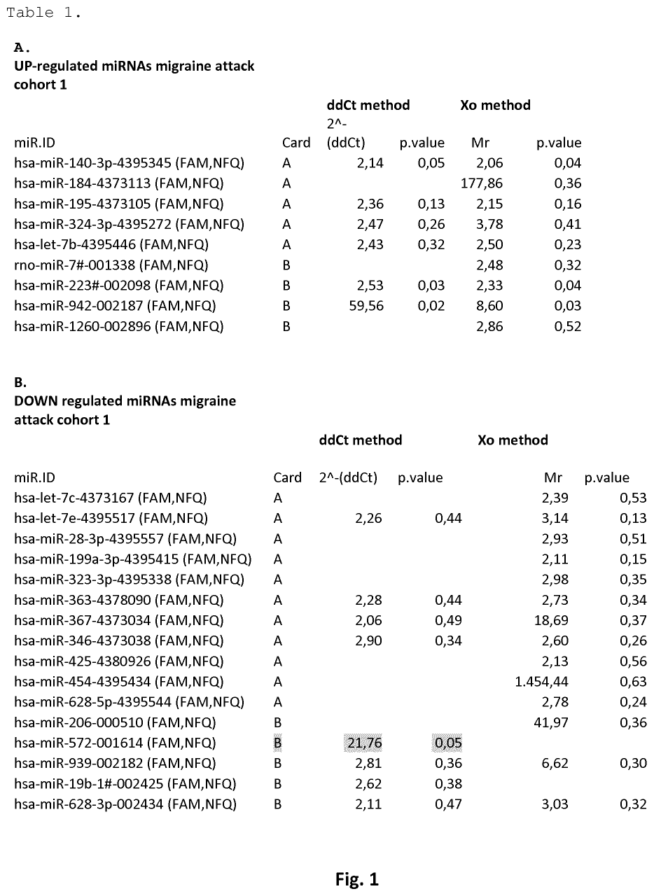

Serum from 8 migraine patients during migraine attack and in pain-free period was screened by using miScript miRNA PCR-array (Qiagen, Hilden, Germany) where the abundance of 372 miRNAs detectable in serum was profiled. The study confirmed an altered miRNA expression during migraine attack compared to a pain-free period (FIG. 1), with a number of miRNAs significantly changed in relative abundance after paired t-tests (p<0.05). Four upregulated miRNAs miR-34a-5p Accession No.: MIMAT0000255, miR-29c-5p Accession No.: MIMAT0003154, miR-382-5p Accession No.: MIMAT0000737, miR-26b-3p, Accession No.: MIMAT0004500) were significantly upregulated.

The four upregulated miRNAs were further validated in a cohort of 12 migraine patients during pain-free period compared to 12 age/sex matched controls. MiR-382-5p was found significantly dysregulated and proposed as a potential diagnostic marker (to recognize migraine from healthy individuals) whereas miR-34a-5p and miR-29c-5p could be novel biomarkers of migraine pain (migraine headache from migraine in remission phase). MiR-382-5p is a brain-specific miRNA, mainly found in neurons and cerebrospinal fluid.

### Serum-Exosomal Biomarkers of Migraine and Migraine Pain Headache

Since three of the upregulated miRNAs identified were most likely packed in exosomes (based on bioinofrmatic analysis), miRNA significantly dysregulated in serum-exosomes in migraine patients during migraine attack were identified. Serum samples from the same cohort 1 of 8 migraine patients were further processed to isolate exosomes by ultracentrifugation for miRNA and proteomic analysis. Analysis of particle size showed a very heterogenic population of particles in 50 nm, 120 nm and >150 nm. The four previously identified dysregulated miRNAs in serum were analyzed by Pick&Mix microRNA PCR plates.

A method of diagnosing migraine in a subject is provided. The method comprising: providing an isolated biological sample obtained from the subject comprising predominantly human exosomes fraction. The inventors discovered that the exosomes are enriched in their content with biological markers according to the invention.

The method further provides determining in said isolated biological sample the level of at least one biomarker expression, providing a control level of said at least one biomarker expression, said control level is a base line level of said biological marker expression in healthy subjects, comparing said level of the at least one biomarker expression with said control level of said at least one biomarker expression, indicating that the subject is likely to have a migraine with an aura when said level of at least one biomarker expression in said isolated biological sample is higher than said control level for said at least one biomarker selected from miR-122 and miR-885-5p, and/or said at least one biomarker expression level in said isolated biological sample is lower than said control level for said at least one biomarker selected from miR-135b and miR-146a and indicating that the subject is unlikely to have a migraine with an aura when said level of at least one biomarker expression is lower than or equal to said control level for said at least one biomarker selected from miR-122 and miR-885-5p, and/or said at least one biomarker expression level in said isolated biological sample is higher than or equal to said control level for said at least one biomarker selected from miR-135b and miR-146a.

MiR-382-5p was identified, but only in migraine patients during pain-free periods, which suggested that the miRNA profile is probably different/enriched in isolated vesicles compared to whole serum.

Thus, serum-exosomes were further analyzed by TaqMan LDA card A+B to screen the abundance of 754 miRNAs. Using paired t-tests, three miRNAs were found significantly upregulated (miR-942, miR-223-5p and miR-140-3p). In-silico predicted target of miR-942 was associated with glial to myelin signalling and miR-223-5p was associated with bipolar disorder and severe migraine (Familial hemiplegic migraine).

In a further embodiment of the method of the invention the migraine attack is migraine attack with an aura.

In a method of diagnosing a migraine in a subject wherein an isolated biological sample obtained from the subject comprising predominantly human exosomes fraction is provided. The biological sample can be processed for the purpose of the invention by, for example, ultracentrifugation followed by Mass Spectrometry, PCR for determining the expression level of the protein or TaqMan® Array screen for miRNAs, whereby the level of at least one biomarker expression is determined in the isolated biological sample.

Further the control level of said at least one biomarker expression is provided, the control level being a base line level of said biological marker expression in healthy subjects. The control level can be predetermined by the same methods as the methods used for determining the expression level.

Further the level of the at least one biomarker expression is compared with the control level of the at least one biomarker expression.

It is indicated that the subject is likely to have a migraine with an aura when said at least one biomarker expression level in said isolated biological sample is higher than said control level for said at least one biomarker selected from miR-122 and miR-885-5p, and/or said at least one biomarker expression level in said isolated biological sample is lower than said control level for said at least one biomarker selected from miR-135b and miR-146a. Surprisingly, these markers were determined to have a different expression pattern in exosomes of the healthy subjects and the migraine patients.

In order to indicate that the subject is likely to have a migraine with an aura expression level in said isolated biological sample of miR-122 is increased compare to the control level at least two time, more preferably at least 2.3 times, miR885 is increased compare to the control level at least 2 times, more preferably at least 2.5 times, miR-135b is decreased at least 2 times, more preferably at least 2.5 times, miR-146a is decreased compare to the control level at least 2 times, preferably at least 4 times, more preferably at least 6 times, more preferably at least 10 times, more preferably at least 15 times, more preferably at least 20 times, more preferably at least 25 times, more preferably at least 30 times, more preferably at least 34.6 times, miR-129-3p is decreased at least two times. If the change in these biological molecules is undetectable or less than two fold it can be indicated that the subject is unlikely to have migraine with an aura.

A difference between the level of the at least one biomarker expression and the reference level or the control level of the at least one biomarker expression is significant as determined by p-value less than 0.05. P value is defined as the probability of obtaining a result equal to or “more extreme” than what was actually observed, when the null hypothesis is true and is well established in the art.

According to another embodiment of the invention the difference between the level of the at least one biomarker expression and the reference level or the control level of the at least one biomarker expression is considered significant when the change in the expression level is at least two-fold.

According to another embodiment of the invention, an isolated biological sample can be blood plasma, serum, urine or saliva. It shall also be understood that any other suitable biological fluids, wherein the proteins or miRNA according to the invention can be detected are also suitable for the purpose.

According to another embodiment of the invention, biological sample comprises extracellular vesicles with the diameter of 50 to 2000 nm, preferably serum exosomes or exosome-like vesicles with a diameter between 30 nm and 150 nm, preferably 100 nm.

Preferably, the subject is human.

According to another aspect of the invention the biological compound according to the invention can be used as molecular biomarkers for the in vitro diagnosis of a migraine in a patient. The biological markers according to the invention are APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34, provided with the reference to NCBI database on FIG. 4, Table 2.

According to another aspect of the invention the biological compound according to the invention can be used as molecular biomarkers for the in vitro diagnosis of migraine attacks in a patient. The biological markers according to the invention are hsa-miR-140-3p-4395345 (FAM,NFQ), hsa-miR-184-4373113 (FAM,NFQ), hsa-miR-195-4373105 (FAM,NFQ), hsa-miR-324-3p-4395272 (FAM,NFQ), hsa-let-7b-4395446 (FAM,NFQ), rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), hsa-miR-10b #-002315 (FAM,NFQ), hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV (P55056), desmocollin-1 (Q08554), desmoplakin (PI5924).

The sequences can be found in http://www.mirbase.org_and the accession numbers are provided above.

Serum proteins of a healthy control and serum exosomes of migraine patients were compared by a Venn diagram FIG. 5A. 42% of the proteins were uniquely expressed in serum exosomes from migraine patients and 38% proteins were uniquely expressed in serum of a healthy control. The three candidate proteins: apolipoprotein C-IV (P55056), desmocollin-1 (Q08554) and desmoplakin (PI5924), were uniquely expressed in migraine patients and differentially expressed during migraine attack, but low in abundance according to their iBAQ rank (from 124-157). Furthermore, the top 10 most abundance proteins in serum exosomes from migraine patients and healthy control serum were compared. The top 4 most abundance in healthy serum (Fi2, B) was also among top 10 in isolated exosome fraction. Albumin had iBAQ rank 4, but was excluded as potential contaminant from protein data of migraine patients. Immunoglobulins and Ig kappa chain C region had high abundance in both serum and serum exosomes.

Three proteins were significantly downregulated during migraine headache attack compared to pain-free period (apolipoprotein C-IV, desmoplakin and desmocollin). They were uniquely expressed in exosomes of migraine patients compared to a baseline in the serum from healthy controls.

### Evaluation of Particle Size Distribution of the Isolated Exosomes

The particle size distribution and concentration of isolated vesicles were determined by NTA of selected samples from the validation experiment. The analysis showed that the isolated particles had a size distribution with a mode range of 112-133 nm and mean values of 146-165 nm (FIG. 6). Sample no. 45 and 47 had unimodal distribution (one clear peak), whereas others had two peaks (bimodal) with unevenly particle size distribution. Furthermore, the concentration of pellet vesicles was from 13.1-32.2×10⁹ particle/mL.

Global miRNA Profile in Serum Exosomes Cohort 2 (Migraine Vs Control)

The Xo method (Thomsen et al, 2010) was applied for relative quantification of miRNAs in serum exosomes of migraine patients and healthy controls. Of the 377 miRNA essays on Card A, 51% were quantifiable (192 of 377) and on card B 21% of the 377 miRNAs were quantifiable (79 of 377). There was no unique expressed miRNA in the groups of migraine patients and healthy controls, and top 10 most abundant miRNAs were the same for both groups, except the order of the ranks. However, complete cases were analyzed so this omits uniquely expressed miRNA from the analysis. Fold changes were displayed by scatterplot and significantly altered miRNAs were determined by two-sided independent t-test. Of the miRNAs assayed on Card A, 62% were down-regulated and 38% up-regulated, whereas the fraction of down/up-regulated miRNAs was more balanced on Card B. In total, 23 miRNAs were significantly deregulated using a p-value of 5%. After Bejamini Hochbergs correction for correction of p-values in multiple tests, 4 miRNAs remained significant (FIG. 7). Those were miR-122 (⬆2.3±0.6), miR-885-5p (⬆2.5±0.6), miR-135b (⬇2.5±0.02) and miR-146a (⬇34.6±0.002.

Stratification of Potential miRNA Biomarkers of Migraine

The total number, and number of unique and shared miRNAs across the 3 studies conducted was analysed. Five candidate miRNAs were identified in serum exosomes of migraine patients compared to healthy controls: miR-146a, miR-885-5p, miR-122, miR-129 and miR-135b.

All candidate miRNAs from the three pain biomarker studies were specific for the different fractions of serum and disease stages when compared across the studies.

In more preferable aspects of the invention the MiR-146a since it had the highest fold of downregulation in migraine patients; MiR-885-5p, since it was found significantly upregulated in migraine patients compared to healthy controls, and MiR-135b: in migraine patients this miRNA was significantly downregulated compared to healthy controls.

Further a method for determining a method of determining a predisposition to develop migraine in a subject is provided. As discovered by the inventors, the miRNA and protein markers according to the invention can be used for determining is the subject is predisposed to develop migraine.

The method comprising: providing an isolated biological sample obtained from the subject. Similar to the aspects discussed above, the biological sample can be but not limited to a blood sample, a serum sample, a saliva sample, a urine sample. Preferably, the biological sample comprises predominantly the serum fraction, enriched with exosomes.

In accordance with the method it is provided determining in said isolated biological sample the level of at least one biomarker expression, providing a control level for said at least one biomarker expression determined as a base line level of said biological marker expression in healthy subjects, comparing said level with said control level for at least one biomarker expression, indicating that the subject is likely to develop migraine if said expression level in said isolated biological sample is higher than said control level for said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34; miR-122 and miR-885-5p and/or said at least one biomarker expression level in said isolated biological sample is lower than said control level for said at least one biomarker selected from miR-135b and miR-146a and indicating that the subject is unlikely to develop migraine if said expression level in said isolated biological sample is equal to or lower than said control level for said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34; miR-122 and miR-885-5p and/or said at least one biomarker expression level in said isolated biological sample is equal to or higher than said control level for said at least one biomarker selected from miR-135b and miR-146a.

The invention is further characterized by the following non-limiting items.

Item 1. A method of diagnosing migraine in a subject comprising:
i) providing an isolated biological sample obtained from the subject,
ii) determining the expression level of at least one biomarker in said isolated biological sample, wherein said at least one biomarker is selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, and IGHV4-34,
iii) providing a control expression level for said at least one biomarker, such as a control level determined as a base line level of said biological marker expression in healthy subjects,
iv) comparing the expression level of said at least one biomarker with said control expression level,
v) indicating that the subject is likely to have migraine when said expression level of said at least one biomarker in said isolated biological sample is higher than said control expression level of said at least one biomarker, or indicating that the subject is unlikely to have migraine when said expression level of at least one biomarker in said isolated biological sample is lower than or equal to said control expression level of said at least one biomarker.

Item 2. The method according to item 1, wherein the biomarker is selected from the group consisting of APOC4, DSC1 and DSP.

Item 3. The method of diagnosing migraine in a subject according to any of the item 1 or 2, wherein the age and/or sex of the healthy subjects are matched with the subjects by age and/or sex.

Item 4. A method of diagnosing migraine attack in a subject having migraine comprising:
i) providing an isolated biological sample obtained from the subject,
ii) determining the expression level of said at least one biomarker in said isolated biological sample,
iii) providing a reference expression level for said at least one biomarker determined as a base level of said biomarker expression in the subject in a pain-free period,
iv) comparing said expression level of at least one biomarker with said reference expression level of said at least one biomarker,
v) indicating that the subject is likely to have a migraine attack when the expression level of at least one biomarker in said isolated biological sample is higher than said reference expression level of said at least one biomarker selected from the group consisting of hsa-miR-140-3p-4395345, hsa-miR-184-4373113, hsa-miR-195-4373105, hsa-miR-324-3p-4395272, hsa-let-7b-4395446, rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), and hsa-miR-10b #-002315 (FAM,NFQ), and/or
the expression level of at least one biomarker in said isolated biological sample is lower than said reference expression level for said at least one biomarker selected from the group consisting of hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV, desmocollin-1, and desmoplakin
or
   indicating that the subject is unlikely to have a migraine attack when the expression level of at least one biomarker in said isolated biological sample is lower than or equal to said reference expression level for said at least one biomarker selected from the group consisting of hsa-miR-140-3p-4395345, hsa-miR-184-4373113, hsa-miR-195-4373105, hsa-miR-324-3p-4395272, hsa-let-7b-4395446, rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), and hsa-miR-10b #-002315 (FAM,NFQ),
      and/or
the expression level of at least one biomarker in said isolated biological sample is higher than or equal to said reference expression level for said at least one biomarker selected from the group consisting of hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV, desmocollin-1, and desmoplakin.

Item 5. The method according to item 4, wherein the biomarker is selected from the group consisting of APOC4, DSC1, and DSP.

Item 6. The method of diagnosing migraine attack in a migraine subject according to any of the item 4 or 5, wherein said migraine attack is migraine attack with an aura.

Item 7. A method of diagnosing a migraine in a subject comprising:
(i) providing an isolated biological sample obtained from the subject comprising a predominantly human exosomes fraction or consisting essentially of human exosomes fraction,
(ii) determining the expression level of at least one biomarker in said isolated biological sample,
(iii) providing a control expression level of said at least one biomarker, such as a control expression level in the form of a base line level of said biological marker expression in healthy subjects,
(iv) comparing said expression level of the at least one biomarker with said control expression level of said at least one biomarker,
(v) indicating that the subject is likely to have a migraine with an aura when said expression level of at least one biomarker in said isolated biological sample is higher than said control expression level of said at least one biomarker, wherein said at least one biomarker is selected from miR-122 and miR-885-5p,
   and/or
   when said at least one biomarker expression level in said isolated biological sample is lower than said control expression level of said at least one biomarker, wherein said at least biomarker is selected from miR-135b, miR-129-3p and miR-146a or indicating that the subject is unlikely to have a migraine with an aura when said expression level of at least one biomarker is lower than or equal to said control expression level of said at least one biomarker, wherein said at least biomarker is selected from miR-122 and miR-885-5p,
   and/or said at least one biomarker expression level in said isolated biological sample is higher than or equal to said control expression level of said at least one biomarker, wherein said at least biomarker is selected from miR-135b, miR-129-3p and miR-146a.

Item 8. A method of determining a predisposition to develop migraine in a subject comprising:
i) providing an isolated biological sample obtained from the subject,
ii) determining in said isolated biological sample the expression level of at least one biomarker,
iii) providing a control expression level of said at least one biomarker expression determined as a base line expression level of said biological marker in healthy subjects,
iv) comparing said biomarker expression level with said control level of at least one biomarker expression,
v) indicating that the subject is likely to develop migraine if said expression level in said isolated biological sample is higher than said control expression level of said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34; miR-122 and miR-885-5p and/or said at least one biomarker expression level in said isolated biological sample is lower than said control expression level of said at least one biomarker selected from miR-135b and miR-146a or indicating that the subject is unlikely to develop migraine if said expression level in said isolated biological sample is equal to or lower than said control expression level of said at least one biomarker selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34; miR-122 and miR-885-5p and/or said at least one biomarker expression level in said isolated biological sample is equal to or higher than said control expression level of said at least one biomarker selected from miR-135b and miR-146a.

Item 9. The method of determining a predisposition to develop migraine in a subject according to item 8, wherein the biomarker is selected from the group consisting of miR-122, miR-146a, miR-122, miR-885-5p, APOC4, DSC1, and DSP.

Item 10. The method according to any one of the preceding items, wherein a difference between said level of said at least one biomarker expression and said control expression level or said reference expression level of said at least one biomarker expression is significant as determined by p-value less than 0.05.

Item 11. The method according to any one of the preceding items, wherein the difference between said expression level of said at least one biomarker and said control expression level or said reference level of said at least one biomarker expression is considered significant when the change in the expression level is at least two-fold.

Item 12. The method according to any one of the preceding items, wherein said isolated biological sample is selected from the group consisting of blood plasma, serum, urine, and saliva.

Item 13. The method according to any one of the preceding items, wherein said biological sample comprises extracellular vesicles of 50-20000 nm, preferably serum exosomes with a diameter between 30 nm and 120 nm, preferably serum exosomes with a diameter of 100 nm.

Item 14. The method according to any one of the preceding items, wherein said subject is a human.

Item 15. The method according to any of the preceding items, wherein said method is an in vitro method.

Item 16. A molecular biomarker for use in diagnosing a migraine in a patient, wherein said molecular biomarker is selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, and IGHV4-34.

Item 17. A molecular biomarker for use in diagnosing a migraine attack in a migraine patient, wherein said molecular biomarker is selected from the group consisting of hsa-miR-140-3p-4395345 (FAM,NFQ), hsa-miR-184-4373113 (FAM,NFQ), hsa-miR-195-4373105 (FAM,NFQ), hsa-miR-324-3p-4395272 (FAM,NFQ), hsa-let-7b-4395446 (FAM,NFQ), rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), hsa-miR-10b #-002315 (FAM,NFQ), hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV, desmocollin-1, and desmoplakin.

### EXAMPLES

### Example 1

### Study Population.

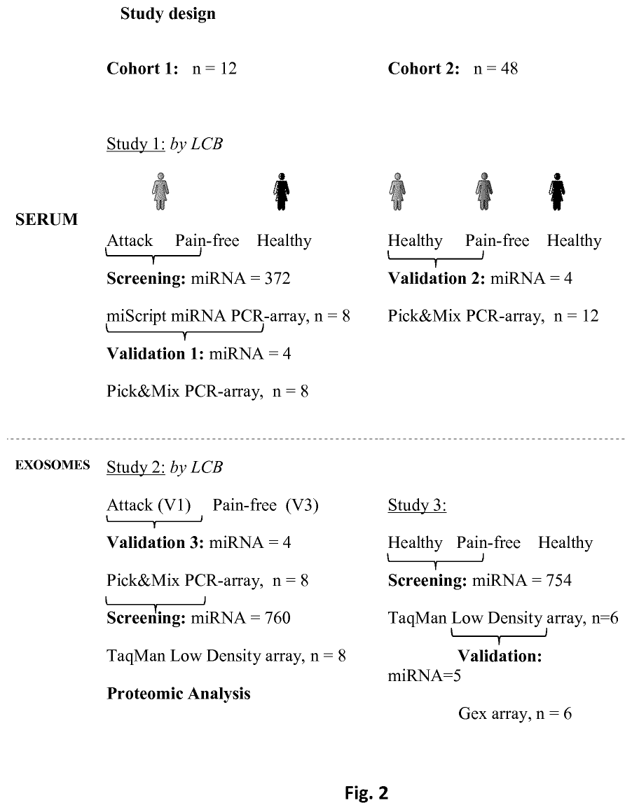

The Study 3, focused on exploring potential diagnostic biomarkers in the exosomal fraction of serum in migraine patients versus healthy age matched controls. The present study followed a general design for studies on miRNAs as potential biomarkers. Hence it included two stages (FIG. 2): 1) screening of dysregulated miRNAs in serum exosomes from 6 migraine patients during pain-free period (cohort 2) and 6 age/sex matched healthy controls, using TaqMan® Array, 2) validation of selected, potential diagnostic miRNAs by GEx Array using serum from the 6 migraine patients and serum from new blood donors as healthy controls.

In study 2, proteomic exosomal data from cohort 1 was processed and analyzed and the exosomal fraction was analysed for miRNA expression to screen for new markers.

Serum samples were obtained post hoc to two previously conducted migraine studies:

Cohort 1: A controlled, parallel study in 2012 investigating potential protein-based biomarkers in migraine patients during attack, remission and pain-free periods. Twelve migraine patients were enrolled (8 females, 4 males, 18-40 years) and 12 sex/age matched healthy volunteers. Serum samples during attack, remission and pain-free periods were collected (Jensen A and Sørensen M, 2012). A cohort subgroup named Cohort 1 was analyzed in Study 1 and 2.

Cohort 2: A randomized, parallel, double-blind, placebo-controlled study of vitamin D as a prophylactic treatment for migraine with 48 enrolled migraine patients and sex/age matched healthy controls in period of 2012-2015 (ClinicalTrials.gov, study nr. N-20120052). Serum samples were collected at baseline (further referred to as pain-free period) before vitamin D treatment and 6 month after the treatment. A cohort subgroup named Cohort 2 was selected for validation purposes (Study 1) and screening/validation in Study 3.

Both studies were approved by the local ethical committee; # N-20110056 (cohort 1) and # N-20120052 (cohort 2). The studies were conducted in accordance with the guidelines of Good Clinical Practice (GCP) from The International Conference on Harmonisation of Technical Requirements for Registration of Pharmaceuticals for Human Use (ICH). Enrolled patients had been diagnosed with chronic migraine according to IHS and common standard procedures were followed for blood collection and serum preparation. Serum samples were stored at −80° C. until analysis or further processing (Jensen A and Sørensen M, 2012; Andersen, 2014).

Controls: Serum samples from 6 blood donors collected in February 2016 at the blood bank, Aalborg University hospital Nord were used as healthy controls (healthy 2) for verification purpose. They were sex and age matched with migraine patients from cohort 2.

### Example 2

### Proteomic Data Analysis.

From cohort 1 files of identified and quantified proteins in the exosomal fraction of 8 migraine patients (6 females, 2 males, avg. age: 28.7, SD: 6.9), were available for further protein analysis. Samples were from migraine patients during headache attack and pain-free period for discovery of pain related, altered proteins. Briefly, isolated serum exosomes were digested proteolyticly into peptides, and triplicates of each digested sample were analyzed using ESI nanoflowLC-MS with label-fee quantitation. The peptides were separated in a high pressure liquid chromatography column (HPLC) before electrospray ionization of peptides and separation based on mass-to-charge ratio. The separated peptides were fragmented into smaller ions followed by a final separation of fragmented peptide ions based on their mass and charge. The amino acid sequences from the mass spectrum of fragments were collected. The raw data files were analysed using MaxQuant (MaxQuant, ver. 1.4.0.288, Max Planck Institute of Biochemistry, Martinsried, Germany).

Protein abundances were presented as protein intensity-based absolute quantitation values, and reported for all proteins having at least two quantifiable peptides (razor+unique) in at least three LC-MS runs.

The samples were searched against the Uniprot human isoform containing reference proteome database [3]. All proteins and peptides were reported at a 1% false discovery rate or better, to ensure only high-confidence protein identifications. The search results were analysed in Perseus (Perseus, ver. 1.4.1.3, Max Planck Institute of Biochemistry, Martinsried, Germany) and GProX (GProX, ver. 1.1.13). The UniProt, C., UniProt: a hub for protein information. Nucleic Acids Res, 2014. 43(Database issue): p. D204-12.

The identified proteins were processed and statistically analyzed in Perseus software ver. 1.5.2.6. The intensity values were log 2 transformed and quantified proteins were additionally filtered, requiring >1 unique peptide in at least 3 samples within a group to reduce the risk of false positive peptides/proteins. Additionally, matching reverse rows were removed together with potential contaminants. The abundance of proteins after filtration was based on the intensity-based absolute quantification (iBAQ). Principle component analysis (PCA) score plot was used to identify and remove replicate outliers. The quantified proteins properties were described using Uniprot gene ontology database. Significant variations in abundant proteins between the two groups (attack vs. pain-free period) were determined by a combined two-sided t-test and fold change. When performing multiple comparisons of proteins with separate hypothesis tests, some would be high or low just by chance even if there is no overall difference. To address this, permutation-based false-positive control was applied (Tusher, 2001) with s0=0.1 and 250 randomizations. The false discovery rate (FDR) was selected to 0.05 allowing 5% to be false positive, e.g. with 200 tests, 190 would be expected to be correct. Significant proteins were those above the cutoff curve (combined p-value and fold change) in the created volcano plot.

The proteins-of-interest were searched in the Exocarta database to verify their exosomal origin and subsequently were compared to proteins of healthy human plasma from another study.

The identified proteins were filtered and evaluated in Perseus software ver. 1.5.2.6. Initially, 278 exosomal proteins were quantified of which 119 proteins were excluded. The technical replicates from one migraine patient (pain-free period) were far from each other, indicating low repeatability. Hence, those data were excluded from further analysis. In total, 159 proteins remained for gene ontology and statistical analysis.

The proteins were functionally characterized based on their associated Gene Ontology terms (www.geneontology.org), Ashburne, 2000), by their biological processes, cellular components and molecular function. The three major biological processes in which the identified proteins were involved were; cellular processes (24%), metabolic processes (21%) and responses to different stimuli (16%). The most prominent stimuli were stress, chemical stimuli and signal transduction. Of cellular components, a large fraction of proteins was from the extracellular environment (68%). However, three of four proteins was associated with blood components e.g. globulins, fibrinogen and proteins of the complement system produced by liver cells, white blood cells or epithelial cells. Only one of four proteins was originated from extracellular vesicles. The major functions of the proteins were binding (30%), especially antigen binding. Additionally, proteins were involved in regulating enzymes involved in binding (18%) and catalytic activities (13%) of binding processes.

### Effect of Migraine Headache on Protein Expression

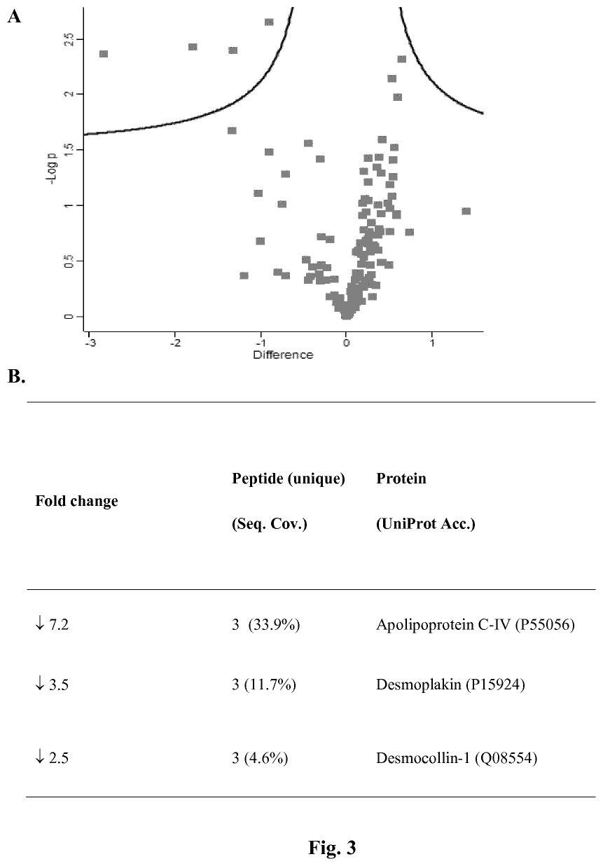

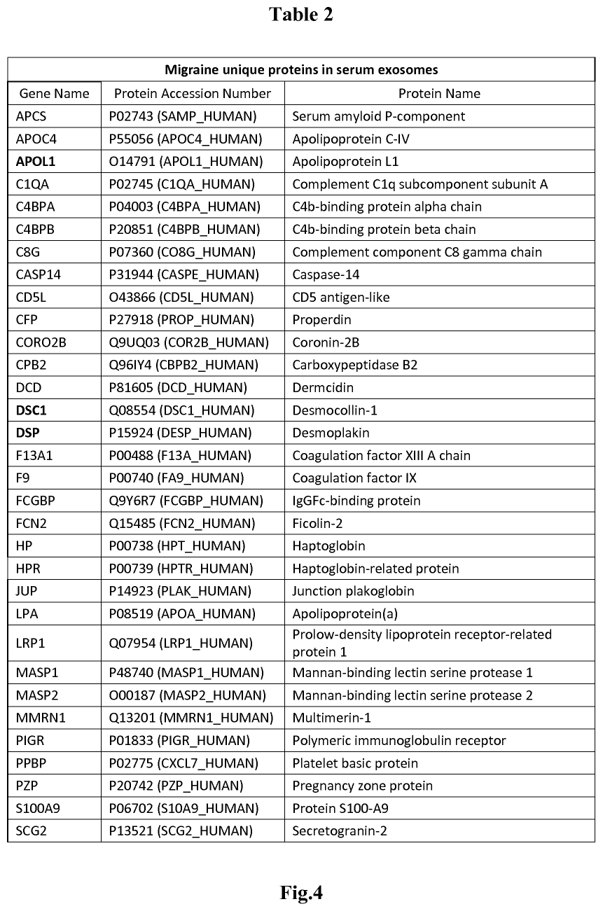

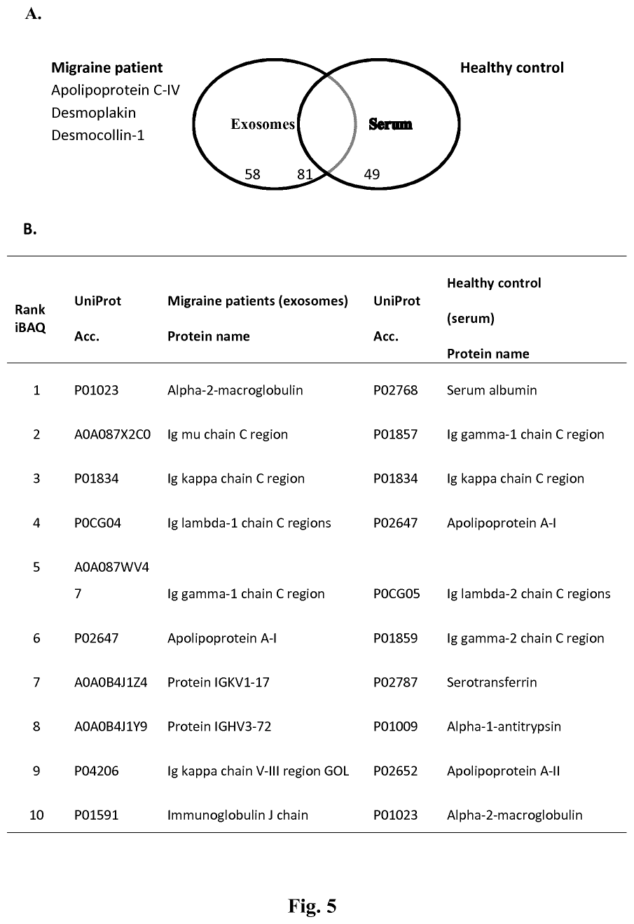

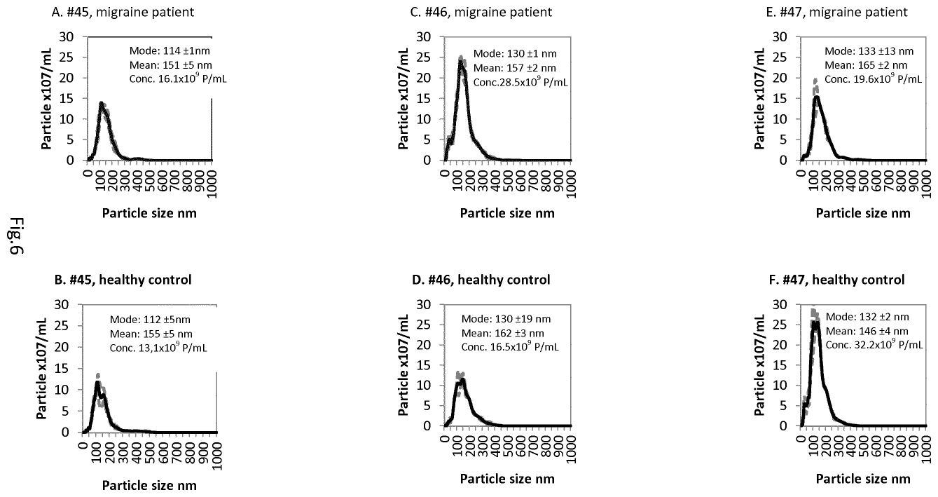

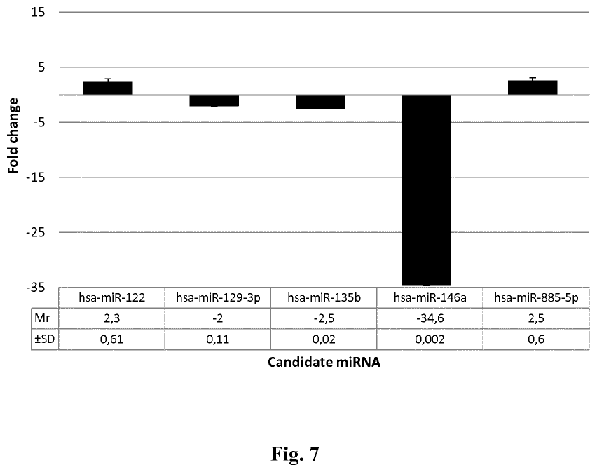

Significantly altered proteins during migraine attack compared to pain-free period were determined by a volcano plot-based strategy in Perseus ver. 1.5.2.6. Three proteins (apolipoprotein C-IV, desmoplakin and desmocollin) were significantly downregulated with more than 2 fold changes, during migraine attack compared to pain-free periods (FIG. 3).

### Example 3

Global Profile of miRNAa in Serum Exosomes: Cohort 2 (Mirgaine Vs Healthy Control)

Isolation of exosomes by ultracentrifugation.

All serum samples for screening and verification purpose (cohort 2 and healthy controls; in total 24 samples) were processed by the following procedure: Thawed serum samples were vortexed for 90 sec to retain all exosomes (Zhou, 2006), then diluted and mixed 1:3 in phosphate buffered saline (PBS), e.g. 0.5 mL serum: 1 mL PBS to reduce viscosity and thereby increase pelleting of vesicles. The diluted samples were centrifuged at 10,000×g for 30 min at 4° C. (Hettich zentrifugen, Universal 320R, Rotor: 1420-A). Supernatant was carefully retained with a hypodermic needle placed opposite to the pellet and filtered through 0.20 μm sterile filter into a 1.5 mL Microfuge® tube Polyallomer (Beckman Instruments, Palo Alto, Calif.) suitable for ultracentrifugation. The samples weights were adjusted with PBS to 2-decimal places and ultracentrifuged for 79 min at 118,000×g, 4° C. (35,000 RPM with LKB 2331 ultrospin 70 Rotor: Thermo Scientific Fiberlite F50L-24×1.5). Supernatant was carefully removed with a hypodermic needle and the pellet vesicles were resuspended in 100 μL PBS before storage at −80° C. Additional samples were processed for NTA analysis. The samples for miRNA screening (12 samples) and verification (12 samples) were sent to AROS Biotechnology A/S, Århus.

### Example 4

### Characterization of Exosomes in Serum, Cohort 2.

Concentration and particle size distribution of the ultracentifuged samples (subset of samples used in the validation experiment) was done using Nanoparticle Tracking Analysis (NTA). The method is a laser scattering microscopically technique, which illuminates particles in liquids to track and capture their movements for size calculation. The particle size can be calculated from a modified stoke-einstein equation, which relates particle movement, viscosity and temperature with the particle size (Gardiner, 2013). The viewing chamber of the nanoparticle visualization system Halo™ LM12 (NanoSight Ltd, Salisbury, UK) was cleaned in 99.9% ethanol and milliQ before the measurements. Samples were thawed/vortexed to retain all vesicles and diluted until a particle concentration was achieved that was between 10⁸/mL-5×10⁹/mL (here 1:50 in DPBS without Ca²⁺ and Mg²⁺). Diluted sample was injected with a 1 mL syringe into the cleaned viewing chamber and mounted on the microscope. The microscope was focused on particles near the fingerprint and a 60-sec video was captured from the moving/light scattered particles with a Marlin F-033B scan camera (ATV, Stadtroda, Germany). Camera level was set to 14 throughout the measurements. Particle concentration and size distribution were calculated from the video of the moving particles analyzed by NTA 2.1 Analytical Software 2010 was used with entered liquid temperature, detection threshold of 6; blur 3×3 and min expected particle size of 50 nm.

### Example 5

### Quantitative Real Time PCR by Taqgan Array

To identify deregulated exosomal miRNA screening was performed by AROS Biotechnology A/S using the medium throughput quantitative real time PCR, TaqMan® Array Human MicroRNA A+B Cards Set v.3.0 (Catalog Number, 4444913, ThermoFischer Scientific). Each card is preconfigured with 384 miRNA assays with endogenous and negative controls, which enable profiling of 754 human miRNAs per sample. The protocol by the manufactures was followed. Briefly, total RNA was purified using miRNeasy Mini Kit from Qiagen. A fixed volume of purified sample (3 μL) was mixed with RT-cDNA master Mix containing Megaplex RT Primer Pools A+B for reverse transcription of mature miRNA species. The cDNA was pre-amplified to increase sensitivity using PreAmp Master Mix with PCR primer, Taqman PreAmp Master Mix and Megaplex PreAmp Primers, Pool A or B. The preamplified product was diluted and added to a PCR mix containing TaqMan Gene Expression Master Mix and loaded into the TagMan Array. Then, it was briefly centrifuged, sealed and analyzed using Applied Biosystems 7900HT Fast Real-Time PCR platform.

### Example 6

### Data Processing

The amplification data were processed in Excel program. Boxplots of the raw data were created to evaluate execution of the experiment. Samples with Ct value distribution far from the other replicates were removed as outliers. Non-detects was replaced with “0” and values >30 were imputed with “30”. The added pre-amplification step reduces the lower acceptable cut off value from 40 to 32 (McDonald). However, the cut off value was selected to 30 in order to compare with the other previous studies (Andersen 2014). All miRNA with less than 3 valid values in a group (migraine patients and healthy controls) were list wise deleted. Remaining non-detects (0 values) were imputed with a random value drawn from a normal distribution the valid values (R. J. A. Little and D. B. Rubin). All cases with 4 valid values in a group (66.6% completed cases) were included in further analysis as processed raw Ct values. The number of quantifiable miRNAs on each card after data processing was calculated without endogenous controls.

### Example 7

### Calculation of Relative Gene Expression by Xo Method and Statistical Analysis.

The relative gene expression was calculated using the Xo method for more precise results. Especially, if there is high variation in target genes compared to controls, the commonly used 2^{-ΔΔCt} method introduces errors in the results, see example in the article by Thomsen et al. (Thomsen et al, 2010). The following procedure was applied for relative quantification of biological replicates with independent relation of target genes: Initially, all Ct values for each miRNAs were converted to linear Xo values (Eq. 1). When assuming 100% amplification efficiency using TaqMan technology (Applied Biosystems) the expression was reduced to: Xo=2^{-Ct}. Then, different normalization methods were tested using Ct values of card A. First, the geometric mean of two selected endogenous controls (by NormFinder, no grouping) was converted to Xo values; then, the endogenous control MammU6 was converted to Xo value, and finally the array mean (equivalent to the global mean) was converted to Xo values. Converted Ct values of each miRNA (Xo,miR) were normalized to the different converted normalizer (Xo,normalizer) by the relative expression Nr (Eq. 2). The average Nr values, N̅r̅ (Eq. 3) were calculated for each group of migraine patients and healthy controls, followed by calculation of standard deviation of N̅r̅ (Eq. 4) and coefficient of variation for N̅r̅ (Eq. 5). The mean CV value for all miRNAs in each group was compared to the mean CV value of non-normalized Ct values (Xo,miR). A lower mean CV value after normalization indicated better capability to reduce experimental induced noise.

The fold difference was expressed as a ratio Mr (Eq. 6) and finally the standard deviation of the fold difference were calculated (Eq. 7).

$$\begin{matrix}Xo = {( 1 + Eamp )}^{- Ct} & \begin{matrix}\text{Conversion of} \text{Ct} \text{values} \\ \text{to linear} \text{Xo} \text{values} \\ Eamp = \text{amplifications efficiency}\end{matrix} & ( Eq . 1 ) \\ Nr = \frac{Xo , miR}{Xo , normalizer} & \begin{matrix}\text{Normalization to} \\ \text{selected endogenous controls,} \\ \text{MammU} \text{6 or the array mean}\end{matrix} & ( Eq . 2 ) \\ \overline{Nr} = \frac{{\sum }_{a}^{i} Nr}{n} & n = \text{number of samples,} a \text{-} i & ( Eq . 3 ) \\ SD ( \overline{Nr} ) = \sqrt{\frac{{\sum }_{a}^{i} {( Nr - \overline{Nr} )}^{2}}{n}} & \begin{matrix}\text{Standard deviation of the} \\ \text{population.} \\ n = \text{number of samples} , a \text{-} i\end{matrix} & ( Eq . 4 ) \\ CV ( \overline{Nr} ) = \frac{SD ( \overline{Nr} )}{\overline{Nr}} & \text{Coefficient of variation} & ( Eq . 5 ) \\ Mr = \frac{\overline{Nr , target}}{\overline{Nr , control}} & Fold change & ( Eq . 6 ) \\ ∓ SD ( Mr ) = Mr \cdot CV ( \overline{Nr} ) & \text{Standard deviation of fold change} & ( Eq . 7 )\end{matrix}$$

### Statistical Analysis

The Nr values were entered in IBM SPSS Statistics v. 22 and test of normality was performed using Shaprio-Wilk Test for small sample size. Multiple independent two sided t-test were performed for Nr values of each miRNA to test significant difference in miRNA expression between migraine patients in pain-free period compared to healthy controls. In order to reduce the rate of false positive values as a result of the high number of tests, the p-values were adjusted by Benjamin-Hochberg: adjusted p-value=r/m·0.05 where r is number of significant miRNA with p-values <0.05 and m is number of tests.

### Example 8

Validation of Identified miRNAs: Cohort 2

For validation of deregulated miRNAs identified during screening, another analysis was performed using other serum samples from cohort 2 (n=6) and new sex/age matched healthy controls (n=6) collected in February 2016. Exosomal miRNAs are stable during storage times of several years, but slight decreases in stability occur during freeze/thaw cycles (Ge, 2014). Thus, the new control samples were kept frozen in −80° C. similar to conditions applied to cohort 2 samples to reduce any differences in miRNA expression due to storage conditions.

### Example 9

### Quantitative Real Time PCR by Fluidigm GEx

Validation of results from TaqMan® Array was performed by AROS Biotechnology A/S, Århus using Fluidigm GEx Array (GE 96×96 Standard v1). TaqMan® Array screen 384 miRNAs per sample, but GEx array enables validation of 96 selected miRNA in up to 96 samples per array, depending on plate formats. The high-throughput method is based on an low volume integrated fluidic circuit on a chip where samples and reagents are mixed through intertwinning channels and sealed by valves in separate compartments with only few nanoliters of the PCR reaction. The significant exosomal miRNAs from the screening experiment were analyzed with two selected reference genes (miR-16, miR-342-3p) based on miRNAs with the lowest stability value according to NormFinder and miRNAs available at AROS.

### Example 10

### Data Processing and Statistical Analysis

Amplification data from the qRT-PCR experiment were processed in Excel and miRNAs with >50% non-detects were excluded. Standard curves of the individual miRNAs showed differences in amplification efficiency (Eamp), which was incorporated in the calculations using the Xo method (Eq. 1). The normalized data (Nr values) were tested for outliers (Eq. 8) and far outliers (Eq. 9) and log transformed to reduced the number of outliers. The log transformed Nr values were tested by Shaprio-Wilks test and found normally distributed. Subsequently, independent two-sided t-test was performed to identify differences in mean Nr values between migraine patients and healthy controls at a significance level of 5%.

| | | |
|---|---|---|
| Outliers: | | |
| | | (Eq. 8) |
| Lower limit: | Q1 = 25% percentile | |
| >Q1 − 1.5 · IQR | Q3 = 75% percentile | |
| Upper limit: | IQR = Q3 − Q1 | |
| <Q1 + 1.5 · IQR | | |
| Far Outliers: | | |
| | | (Eq. 9) |
| Lower limit: | Q1 = 25% percentile | |
| >Q1 − 3 · IQR | Q3 = 75% percentile | |
| Upper limit: | IQR = Q3 − Q1 | |
| <Q1 + 3 · IQR | | |

### Example 10

Stratification of Potential miRNA Biomarkers of Migraine

In order to compare the miRNA expression in the 3 pain biomarker studies, the data from Study 2 were processed with a lower cutoff value of 30, which had been used in Study 1+3. The data from Study 1 and 2 were processed as described in the section “Data processing” and total number of identified miRNAₛ, number of shared and unique miRNAs was registered for the 3 studies in Excel program.

### Example 11

In Silico miRNA Target Prediction

The candidate miRNAs were entered in the freely accessible database of miRWalk2.0 (http://zmf.umm.uni-heidelberg.de), which provides information on predicted and validated targets of human, rat and mouse miRNAs. The predicted target module “MicroRNA-gene Targets” was selected with standard settings (candidate miRNA should be present in all four databases miRWalk, RNA22, miRanda and Targetscan) and minimum seed length was adjusted from 7 to 10 and adjusted p-value from 0.05 to 0.001 to filter and reduce the list of possible hits. Top 10 matches of putative target genes found in selected databases were evaluated based on a summary of predicted functions from NCBI gene provided by the RefSeq (NCBI Reference Sequences). The predicted function of a candidate miRNA is referred to by gene symbol and RefseqIlD.

### REFERENCES

Andersen, H H; Gazerani, P; Duroux, M. EHMTI-0122. Serum micrornas as potential biomarkers of migraine. The Journal of Headache and Pain, 2014, Vol. 15(1), pp. 1-1.
Colombo, Marina; Raposo, Graa; Thry, Clotilde. Biogenesis, Secretion, and Intercellular Interactions of Exosomes and Other Extracellular Vesicles. Annual Review of Cell and Developmental Biology, 2014, Vol. 30, p. 255-289
Costa F, Alexandra L, Neto F, Lourença M (2015) Satellite glial cells in sensory ganglia: its role in pain. Brazilian Journal of Anesthesiology (English edition), Vol. 65(1):73-81. DOI: 10.1016/j.bjane.2013.07.013
Etheridge A, Lee I, Hood L, Galas D, Wang K (2011). Extracellular microRNA: a new source of biomarkers. Mutat Res.; 717(1-2): 85-90. doi:10.1016/j.mrfmmm.2011.03.004.
Gupta A, Pulliam L (2014) Exosomes as mediators of neuroinflammation. Journal of neuroinflammation, Vol. 11:68. DOI: 10.1186/1742-2094-11-68
Jasmin L, Vit J p, Bhargava A, Ohara P T (2010) Can satellite glial cells be therapeutic targets for pain control? Neuron Glia Biology 6(1):63-71. doi: 10.1017/S1740925X10000098
Ji R R, Berta T, Nedergaard M (2013) Glia and pain: Is chronic pain a gliopathy?. Pain 154:S10-S28. doi: 10.1016/j.pain.2013.06.022
Kojić Z, Stojanović D (2013) Pathophysiology of migraine—from molecular to personalized medicine. Medicinski pregled, Vol. 66(1-2): 53-7.
Kynast K, Russe O Q, Geisslinger G, Niederberger E (2013) Novel findings in pain processing pathways: implications for miRNAs as future therapeutic targets. Expert Rev. Neurother. 13(5),
Loder E, Biondi D (2005) General Principles of Migraine Management: The Changing Role of Prevention. Headache: The Journal of Head and Face Pain, Vol. 45, pp. S33-S47. DOI:10.1111/j.1526-4610.2005.4501002.x
MiRWalk 2.0. a comprehensive atlas of predicted and validated miRNA-target interactions. http://zmf.umm.uni-heidelberg.de/apps/zmf/mirwalk2/
NormFinder version 2.0. Documentation for NormFinder.http://moma.dk/files/Normfinder %20documentation-v20.pdf. Accessed 20. January 2016.
Ohara P T, Vit J P, Bhargava A et al (2009) Gliopathic Pain: When Satellite Glial Cells Go Bad. The Neuroscientist 15(5):450-463. doi: 10.1177/1073858409336094
Russell M B, Ulrich V, Gervil M, Olesen J (2002) Migraine Without Aura and Migraine With Aura Are Distinct Disorders. A Population-Based Twin Survey. Headache: The Journal of Head and Face Pain, Vol. 42(5): 332-336. DOI: 10.1046/j.1526-4610.2002.02102.x
Tafuri E et al. (2015) MicroRNA profiling in migraine without aura: Pilot study. Annals of medicine (Helsinki), p. 1-6.
Thomsen R, Sølvsten CAE, Linnet T E, Blechingberg J, Nielsen A L (2010) Analysis of qPCR data by converting exponentially related Ct values into linearly related X0 values. Journal of bioinformatics and computational biology, Vol. 8(5): 885-900.

1. A method of evaluating a migraine in a subject in need thereof comprising:
i) providing an isolated biological sample from the subject,
ii) determining in said isolated biological sample the level of expression of at least one biomarker,
iii) providing a control level of expression for said at least one biomarker determined as a base line level of expression of said at least one biomarker in healthy subjects,
iv) comparing said level of expression of said at least one biomarker with said control level of expression of said at least one biomarker, and
v) indicating that the subject is likely to have a migraine when said level of expression of said at least one biomarker in said isolated biological sample is higher than said control level of expression for said at least one biomarker, wherein said at least one biomarker is selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, and IGHV4-34, or
vi) indicating that the subject is unlikely to have a migraine when said level of expression of said at least one biomarker in said isolated biological sample is lower than or equal to said control level of expression for said at least one biomarker, wherein said at least one biomarker is selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, and IGHV4-34.

2-17. (canceled)

18. The method according to claim 1, wherein the at least one biomarker is selected from the group consisting of APOC4, DSC1, and DSP.

19. The method according to claim 1, wherein the healthy subjects are matched with the subjects in need thereof by age and/or sex.

20. A method of diagnosing a migraine attack in a subject having a migraine comprising:
i) providing an isolated biological sample obtained from the subject,
ii) determining in said isolated biological sample the level of expression of at least one biomarker,
iii) providing a reference level for said expression of said at least one biomarker, determined as a base level of expression of said biomarker in the subject in a pain-free period,
iv) comparing said level of expression of said at least one biomarker with said reference level of expression of said at least one biomarker, and
v) indicating that the subject is likely to have a migraine attack when:
the expression level of said at least one biomarker in said isolated biological sample is higher than said reference level of expression for said at least one biomarker, wherein said biomarker is selected from the group consisting of hsa-miR-140-3p-4395345, hsa-miR-184-4373113, hsa-miR-195-4373105, hsa-miR-324-3p-4395272, hsa-let-7b-4395446, rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), and hsa-miR-10b #-002315 (FAM,NFQ),
and/or
the level of expression of at least one biomarker in said isolated biological sample is lower than said reference level of expression for said at least one biomarker, wherein said at least one biomarker is selected from the group consisting of hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV, desmocollin-1, and desmoplakin
or
vi) indicating that the subject is unlikely to have a migraine attack when the level of expression of said at least one biomarker in said isolated biological sample is lower than or equal to said reference level of expression for said at least one biomarker, wherein said at least one biomarker is selected from the group consisting of hsa-miR-140-3p-4395345, hsa-miR-184-4373113, hsa-miR-195-4373105, hsa-miR-324-3p-4395272, hsa-let-7b-4395446, rno-miR-7#-001338 (FAM,NFQ), hsa-miR-223#-002098 (FAM,NFQ), hsa-miR-942-002187 (FAM,NFQ), hsa-miR-1260-002896 (FAM,NFQ), hsa-miR-34a-4395168 (FAM,NFQ), hsa-miR-185-4395382 (FAM,NFQ), hsa-miR-193a-5p-4395392 (FAM,NFQ), hsa-miR-224-4395210 (FAM,NFQ), hsa-miR-340-4395369 (FAM,NFQ), hsa-miR-522-4395524 (FAM,NFQ), hsa-miR-579-4395509 (FAM,NFQ), hsa-miR-511-4373236 (FAM,NFQ), dme-miR-7-000268 (FAM,NFQ), and hsa-miR-10b #-002315 (FAM,NFQ),
and/or
the level of at least one expression of said at least one biomarker in said isolated biological sample is higher than or equal to said reference level of expression for said at least one biomarker, wherein said at least one biomarker is selected from the group consisting of hsa-let-7c-4373167 (FAM,NFQ), hsa-let-7e-4395517 (FAM,NFQ), hsa-miR-28-3p-4395557 (FAM,NFQ), hsa-miR-199a-3p-4395415 (FAM,NFQ), hsa-miR-323-3p-4395338 (FAM,NFQ), hsa-miR-363-4378090 (FAM,NFQ), hsa-miR-367-4373034 (FAM,NFQ), hsa-miR-346-4373038 (FAM,NFQ), hsa-miR-425-4380926 (FAM,NFQ), hsa-miR-454-4395434 (FAM,NFQ), hsa-miR-628-5p-4395544 (FAM,NFQ), hsa-miR-206-000510 (FAM,NFQ), hsa-miR-572-001614 (FAM,NFQ), hsa-miR-939-002182 (FAM,NFQ), hsa-miR-19b-1#-002425 (FAM,NFQ), hsa-miR-628-3p-002434 (FAM,NFQ), apolipoprotein C-IV, desmocollin-1, and desmoplakin.

21. The method according to claim 20, wherein the at least one biomarker is selected from the group consisting of APOC4, DSC1, and DSP.

22. The method according to claim 20, wherein said migraine attack is migraine attack with an aura.

23. A method of diagnosing a migraine in a subject in need thereof comprising:
i) providing an isolated biological sample obtained from the subject comprising exosomes,
ii) determining in said isolated biological sample the level of expression of at least one biomarker,
iii) providing a control level of expression of said at least one biomarker expression, said control level being a base line level of said biological marker expression in healthy subjects,
iv) comparing said level of expression of the at least one biomarker with said control level of expression of said at least one biomarker,
v) indicating that the subject is likely to have a migraine with an aura when said level of expression of said at least one biomarker in said isolated biological sample is higher than said control level of expression for said at least one biomarker, wherein said biomarker is selected from miR-122 or miR-885-5p,
and/or
said expression level of said at least one biomarker in said isolated biological sample is lower than said control level of expression for said at least one biomarker, wherein said at least one biomarker is selected from miR-135b, miR-129-3p or miR-146a
and indicating that the subject is unlikely to have a migraine with an aura when said level of expression of said at least one biomarker is lower than or equal to said control level of expression for said at least one biomarker, wherein said at least one biomarker is selected from miR-122 or miR-885-5p,
and/or said expression level of said at least one biomarker in said isolated biological sample is higher than or equal to said control level of expression for said at least one biomarker, wherein said at least one biomarker is selected from miR-135b, miR-129-3p or miR-146a.

24. A method of determining a predisposition to develop migraine in a subject in need thereof comprising:
i) providing an isolated biological sample obtained from the subject,
ii) determining in said isolated biological sample the level of expression of at least one biomarker,
iii) providing a control level of expression for said at least one biomarker determined as a base line level of expression of said at least one biomarker in healthy subjects,
iv) comparing said level of expression of said at least one biomarker in the isolated biological sample with said control level of expression of said at least one biomarker,
v) indicating that the subject is likely to develop migraine if said expression level of said at least one biomarker in said isolated biological sample is higher than said control level of expression for said at least one biomarker, wherein said at least one biomarker is selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34; miR-122 and miR-885-5p and/or said expression level of said at least one biomarker in said isolated biological sample is lower than said control level of expression for said at least one biomarker, wherein said at least one biomarker is selected from miR-135b or miR-146a and indicating that the subject is unlikely to develop migraine if said expression level of said at least one biomarker in said isolated biological sample is equal to or lower than said control level for said at least one biomarker, wherein said at least one biomarker is selected from the group consisting of APCS, APOC4, APOL1, C1QA, C4BPA, C4BPB, C8G, CASP14, CD5L, CFP, CORO2B, CPB2, DCD, DSC1, DSP, F13A1, F9, FCGBP, FCN2, HP, HPR, GHV3-30, IGHV3-49, IGHV3-72, IGHV3-74, IGHV4-34; miR-122 and miR-885-5p and/or said expression level of said at least one biomarker in said isolated biological sample is equal to or higher than said control level of expression for said at least one biomarker, wherein said at least one biomarker is selected from miR-135b or miR-146a.

25. The method according to claim 24, wherein the biomarker is selected from the group consisting of miR-122, miR-146a, miR-122, miR-885-5p, APOC4, DSC1, and DSP.

26. The method according to claim 24, wherein a difference between said level of expression of said at least one biomarker and said control level of expression of said at least one biomarker expression is significant as determined by p-value less than 0.05.

27. The method according to claim 24, wherein the difference between said level of expression of said at least one biomarker and said control level of expression of said at least one biomarker is considered significant when the change in the expression level is at least two-fold.

28. The method according to claim 1, wherein said isolated biological sample is selected from the group consisting of blood plasma, serum, urine, and saliva.

29. The method according to claim 23, wherein said biological sample comprises extracellular vesicles of 50-20000 nm, or serum exosomes with a diameter between 30 nm and 120 nm.

30. The method according to claim 1, wherein said subject is a human.

31. The method according to claim 1, wherein said method is an in vitro method.
